(19) United States
(12) Patent Application Publication
(10) Pub. No.: US 2024/0225506 A1
(43) Pub. Date: Jul. 11, 2024

(54) DENTAL PROSTHETIC DEVICE AND METHOD OF FORMING SAME

(71) Applicant: NEOCIS INC., Miami, FL (US)

(72) Inventors: Alon Mozes, Miami Beach, FL (US); Dennis Moses, Hollywood, FL (US); Erik Paul Flor Beuckelaers, Miami Shores, FL (US)

(21) Appl. No.: 18/563,079

(22) PCT Filed: May 24, 2022
(86) PCT No.: PCT/IB2022/054854

§ 371 Date: Nov. 21, 2023

Related U.S. Application Data
(60) Provisional application No. 63/194,791, filed on May 28, 2021.

(51) Int. Cl. A61B 5/22; A61B 5/00; A61C 8/00; A61B 90/00

(52) U.S. Cl. CPC A61B 5/228; A61B 5/4851; A61B 5/682; A61C 8/0093; A61B 2090/064; A61B 2560/0219

(57) ABSTRACT

A dental prosthetic device includes at least an anchor portion and in some instances a prosthetic portion arranged to engage the anchor portion. A sensor device is arranged to be engaged with the anchor portion. When the dental prosthetic device includes a prosthetic portion, the sensor device is arranged to be engaged with the anchor portion, with the prosthetic portion, or between the anchor portion and the prosthetic portion. A method of forming a dental prosthetic device is also provided.

### BACKGROUND

### Field of the Disclosure

The present disclosure relates to dental prosthetic devices and, more particularly, to a dental implant or portion thereof, and other dental prosthetic devices, including one or more sensors for monitoring the dental implant, the portion of the dental implant, or the other dental prosthetic device.

### Description of Related Art

Dental implants 10 are typically simple, non-electronic devices, including an anchor portion 20 (in some instances, comprising two or more components such as a screw portion and an abutment received by the screw portion) made of titanium or some other standard biocompatible material, and a prosthetic portion 30 (e.g., a crown) received by the anchor portion 20 (or the abutment component thereof) made of metal (such as stainless steel, gold, or an alloy), porcelain-fused-to-metal, a resin, or a ceramic (see, e.g., FIG. 1). Once implanted, the intent is for the anchor portion to properly osseointegrate into the patient's jaw, which can be indicated by pressure around/on the anchor portion of the dental implant. Moreover, the anchor portion is usually a threaded member that interacts with a bore in the jawbone to threadedly engaged and secure the anchor portion to the jaw. As such, proper osseointegration and/or securement of the anchor portion the jawbone could also be indicated by sufficient pressure on the threads of the anchor portion.

The implanted dental implant may also be subjected to various forces during the service life of the implant. For example, the dental implant may experience forces from chewing, grinding/bruxation at night, jaw clenching, etc. In some instances, the magnitude of the force(s) may damage the dental implant (the prosthetic portion, the anchor portion, the interface between the anchor portion and the prosthetic portion and/or the interface between the anchor portion and the jawbone). In other instances, repeated cycles of force(s) of a certain magnitude may lead to fatigue damage to the prosthetic portion, the anchor portion, the interface between the anchor portion and the prosthetic portion, and/or the interface between the anchor portion and the jawbone. In instances of damage to the implanted dental implant, remedying of the damage often involves entire replacement of the dental implant.

As such, there exists a need for a dental implant capable of indicating whether the dental implant has been properly implanted in the patient's jawbone, and whether the patient's jawbone has accepted and properly osseointegrated the anchor portion. Moreover, there exists a need for a dental implant capable of indicating the various force(s) experienced by the dental implant in service such that possible damage to the dental implant can be investigated and remedied prior to failure. In addition, there exists a need for dental implant capable of monitoring force(s) and force cycle(s) experienced by the dental implant such that remedial action before failure can be taken prior to the dental implant reaching fatigue limits or end of service life.

### SUMMARY

The above and other needs are met by aspects of the present disclosure which includes, without limitation, the following example embodiments and, in one particular aspect, provides a dental prosthetic device, comprising an anchor portion and a sensor device arranged to be engaged with the anchor portion.

Another aspect of the present disclosure provides a method of forming a dental prosthetic device, comprising engaging a sensor device with an anchor portion of a dental prosthetic.

The present disclosure thus includes, without limitation, the following example embodiments:

Example Embodiment 1: A dental prosthetic device, comprising an anchor portion; and a sensor device arranged to be engaged with the anchor portion.

Example Embodiment 2: The device of any preceding example embodiment, or combinations thereof, comprising a prosthetic portion arranged to engage the anchor portion, wherein the sensor device is arranged to be engaged with the anchor portion, with the prosthetic portion, or between the anchor portion and the prosthetic portion.

Example Embodiment 3: The device of any preceding example embodiment, or combinations thereof, wherein the sensor device is a force sensor.

Example Embodiment 4: The device of any preceding example embodiment, or combinations thereof, wherein the sensor device is a force sensor, and wherein the force sensor is disposed between the anchor portion and the prosthetic portion, within the prosthetic portion, or on a surface of the prosthetic portion, and is arranged to measure a force imparted to the prosthetic portion.

Example Embodiment 5: The device of any preceding example embodiment, or combinations thereof, wherein the anchor portion defines external threads adapted to threadedly engage a structure, and wherein the force sensor is engaged with the external threads and is arranged to measure a threading force engaging the anchor portion with the structure.

Example Embodiment 6: The device of any preceding example embodiment, or combinations thereof, wherein the anchor portion includes a wall defining an internal channel, and wherein the force sensor is engaged with the wall within the internal channel and is arranged to measure forces imparted to the anchor portion.

Example Embodiment 7: The device of any preceding example embodiment, or combinations thereof, wherein the sensor device is a contact sensor engaged with a surface of the prosthetic portion and is arranged to sense external contact with the prosthetic portion.

Example Embodiment 8: The device of any preceding example embodiment, or combinations thereof, comprising a power source arranged in electrical communication with the sensor device, and disposed within the anchor portion.

Example Embodiment 9: The device of any preceding example embodiment, or combinations thereof, comprising a power source arranged in electrical communication with the sensor device, and disposed within the anchor portion, within the prosthetic portion, or between the anchor portion and the prosthetic portion.

Example Embodiment 10: The device of any preceding example embodiment, or combinations thereof, comprising a power source arranged in electrical communication with the sensor device, wherein the power source is arranged to generate electrical power in response to vibration or movement thereof.

Example Embodiment 11: The device of any preceding example embodiment, or combinations thereof, comprising a transmitter in communication with the sensor device and with the power source, wherein the transmitter is arranged to be powered by the power source to transmit data from the sensor device away from the dental prosthetic.

Example Embodiment 12: The device of any preceding example embodiment, or combinations thereof, comprising a prosthetic portion arranged to engage the anchor portion, wherein the transmitter is engaged with the anchor portion, with the prosthetic portion, or between the anchor portion and the prosthetic portion.

Example Embodiment 13: The device of any preceding example embodiment, or combinations thereof, comprising a transmitter in communication with the sensor device, wherein the transmitter is arranged to implement a contactless communication modality to transmit data from the sensor device away from the dental prosthetic.

Example Embodiment 14: The device of any preceding example embodiment, or combinations thereof, comprising a reservoir arranged to contain a medicament, and a dispensing device in communication with the reservoir and arranged to dispense the medicament therefrom away from the dental prosthetic.

Example Embodiment 15: The device of any preceding example embodiment, or combinations thereof, comprising a prosthetic portion arranged to engage the anchor portion, wherein each of the reservoir and the dispensing device is arranged within the anchor portion, within the prosthetic portion, or between the prosthetic portion and the anchor portion.

Example Embodiment 16: A method of forming a dental prosthetic device, comprising engaging a sensor device with an anchor portion of a dental prosthetic.

Example Embodiment 17: The method of any preceding example embodiment, or combinations thereof, wherein the dental prosthetic includes a prosthetic portion arranged to be engageable with the anchor portion, and wherein the method comprises engaging the sensor device with the anchor portion, with the prosthetic portion, or between the prosthetic portion and the anchor portion.

Example Embodiment 18: The method of any preceding example embodiment, or combinations thereof, wherein engaging the sensor device comprises engaging a force sensor with the anchor portion.

Example Embodiment 19: The method of any preceding example embodiment, or combinations thereof, wherein engaging the sensor device comprises engaging a force sensor between the anchor portion and the prosthetic portion, within the prosthetic portion, or on a surface of the prosthetic portion, such that the force sensor is arranged to measure a force imparted to the prosthetic portion.

Example Embodiment 20: The method of any preceding example embodiment, or combinations thereof, wherein the anchor portion defines external threads adapted to threadedly engage a structure, and wherein engaging the sensor device comprises engaging the force sensor with the external threads such that the force sensor is arranged to measure a threading force engaging the anchor portion with the structure.

Example Embodiment 21: The method of any preceding example embodiment, or combinations thereof, wherein the anchor portion includes a wall defining an internal channel, and wherein engaging the sensor device comprises engaging the force sensor with the wall within the internal channel such that the force sensor is arranged to measure forces imparted to the anchor portion.

Example Embodiment 22: The method of any preceding example embodiment, or combinations thereof, wherein engaging the sensor device comprises engaging a contact sensor with a surface of the prosthetic portion such that the contact sensor is arranged to sense external contact with the prosthetic portion.

Example Embodiment 23: The method of any preceding example embodiment, or combinations thereof, comprising arranging a power source within the anchor portion such that the power source is in electrical communication with the sensor device.

Example Embodiment 24: The method of any preceding example embodiment, or combinations thereof, comprising arranging a power source within the anchor portion, within the prosthetic portion, or between the anchor portion and the prosthetic portion, such that the power source is in electrical communication with the sensor device.

Example Embodiment 25: The method of any preceding example embodiment, or combinations thereof, comprising arranging a power source in electrical communication with the sensor device, wherein the power source is arranged to generate electrical power in response to vibration or movement thereof.

Example Embodiment 26: The method of any preceding example embodiment, or combinations thereof, comprising arranging a transmitter in communication with the sensor device and with the power source, wherein the transmitter is arranged to be powered by the power source to transmit data from the sensor device away from the dental prosthetic.

Example Embodiment 27: The method of any preceding example embodiment, or combinations thereof, wherein the dental prosthetic includes a prosthetic portion arranged to engage the anchor portion, and wherein arranging the transmitter comprises arranging the transmitter in engagement with the anchor portion, with the prosthetic portion, or between the anchor portion and the prosthetic portion.

Example Embodiment 28: The method of any preceding example embodiment, or combinations thereof, comprising engaging a transmitter in communication with the sensor device, wherein the transmitter is arranged to implement a contactless communication modality to transmit data from the sensor device away from the dental prosthetic.

Example Embodiment 29: The method of any preceding example embodiment, or combinations thereof, comprising engaging a reservoir arranged to contain a medicament, and a dispensing device in communication with the reservoir and arranged to dispense the medicament therefrom away from the dental prosthetic, with the anchor portion.

Example Embodiment 30: The method of any preceding example embodiment, or combinations thereof, wherein the dental prosthetic includes a prosthetic portion arranged to engage the anchor portion, and wherein the method comprises arranging each of the reservoir and the dispensing device within the anchor portion, within the prosthetic portion, or between the prosthetic portion and the anchor portion.

These and other features, aspects, and advantages of the present disclosure will be apparent from a reading of the following detailed description together with the accompanying drawings, which are briefly described below. The present disclosure includes any combination of two, three, four, or more features or elements set forth in this disclosure, regardless of whether such features or elements are expressly combined or otherwise recited in a specific embodiment description herein. This disclosure is intended to be read holistically such that any separable features or elements of the disclosure, in any of its aspects and embodiments, should be viewed as intended, namely to be combinable, unless the context of the disclosure clearly dictates otherwise.

It will be appreciated that the summary herein is provided merely for purposes of summarizing some example aspects so as to provide a basic understanding of the disclosure. As such, it will be appreciated that the above described example aspects are merely examples and should not be construed to narrow the scope or spirit of the disclosure in any way. It will be appreciated that the scope of the disclosure encompasses many potential aspects, some of which will be further described below, in addition to those herein summarized. Further, other aspects and advantages of such aspects disclosed herein will become apparent from the following detailed description taken in conjunction with the accompanying drawings which illustrate, by way of example, the principles of the described aspects.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIG. 1 schematically illustrates a prior art example of a dental prosthetic;

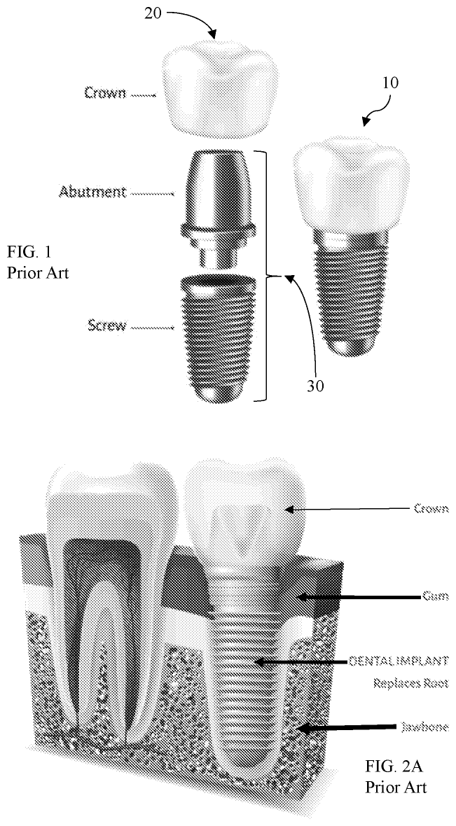

FIGS. 2A-2C schematically illustrate different prior art forms of a dental prosthetic;

FIG. 3 schematically illustrates a prior art example of a film-type sensor device;

FIG. 4 schematically illustrates a dental prosthetic device, according to one aspect of the present disclosure, implementing one or more sensor devices; and

FIG. 5 schematically illustrates a dental prosthetic device, according to other aspects of the present disclosure, implementing one or more sensor devices.

### DETAILED DESCRIPTION

The present disclosure now will be described more fully hereinafter with reference to the accompanying drawings, in which some, but not all aspects of the disclosure are shown. Indeed, the disclosure may be embodied in many different forms and should not be construed as limited to the aspects set forth herein; rather, these aspects are provided so that this disclosure will satisfy applicable legal requirements. Like numbers refer to like elements throughout.

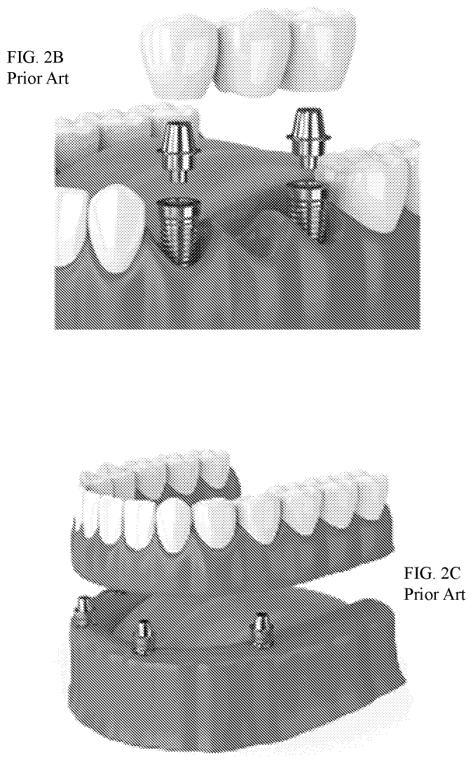

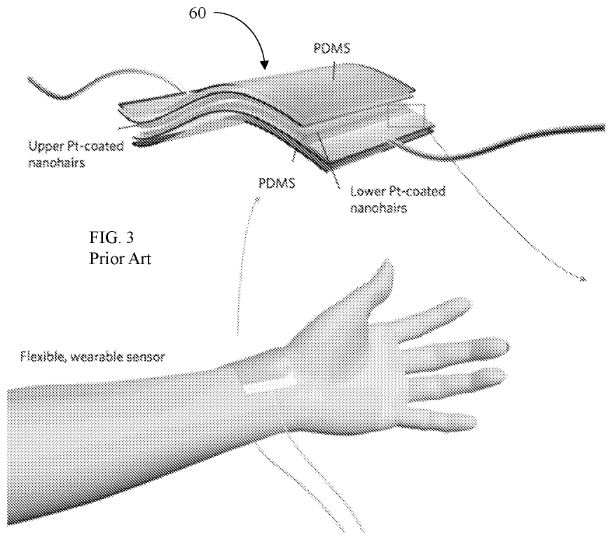

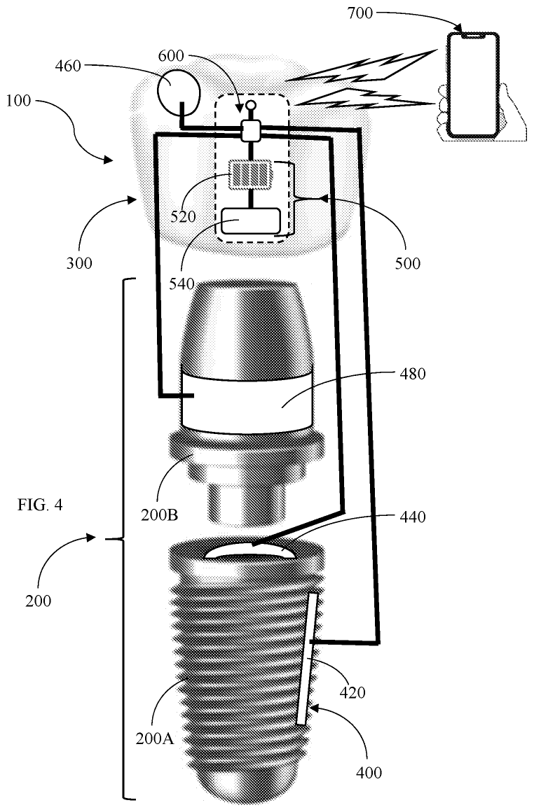

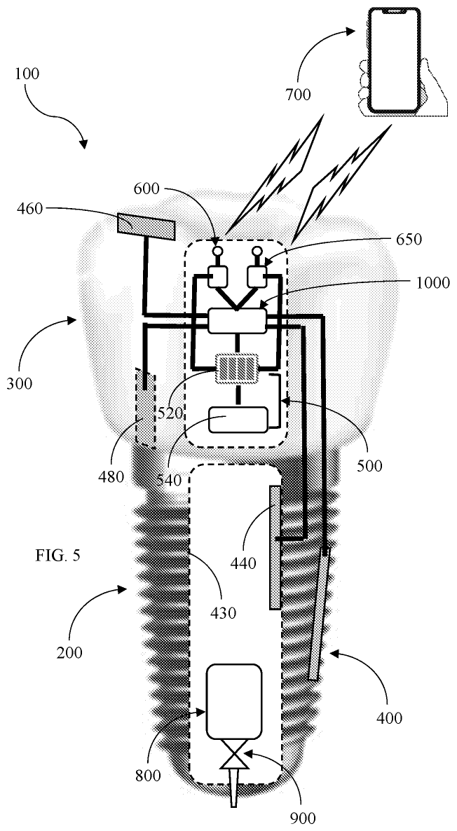

FIGS. 4 and 5 schematically illustrates a dental prosthetic device 100 according to one aspect of the present disclosure. The dental prosthetic device 100 comprises an anchor portion 200 and a prosthetic portion 300 arranged to engage the anchor portion 200. Such a dental prosthetic device 100 may comprise, for example, a dental implant including an anchor post (anchor portion 200) comprising a screw portion 200A capable of being implanted into (e.g., threadedly engaging) the jaw of a patient, an abutment 200B received by the screw portion 200A and serving as an interface for engaging a crown (prosthetic portion 300), wherein the crown is thus received by and secured to the anchor post. In other aspects of the disclosure, such a dental prosthetic device 100 can also include, for example, implants, an implants bar, abutments, overdenture prosthetics, and/or other dental devices classified as a dental prosthetic, and/or combinations thereof, generally including an implantable anchor portion and an aesthetic aspect comprising the prosthetic portion (see, e.g., examples of dental prosthetic in the form of a single tooth dental implant, an implant-supported bridge, and an implant-supported denture, shown in FIGS. 2A, 2B, and 2C, respectively). In aspects of the disclosure, a sensor device 400 (see, e.g., an example of a film-type sensor 60 shown in FIG. 3) is arranged to be engaged with at least the anchor portion 200. In other aspects, the sensor device 400 is arranged to be engaged with the anchor portion 200, with the prosthetic portion 300, or between the anchor portion 200 and the prosthetic portion 300.

In some aspects, the sensor device 400 is, for example, a force sensor. In particular aspects, the force sensor is disposed between the anchor portion 200 and the prosthetic portion 300, within the prosthetic portion 300, or on a surface of the prosthetic portion 300. In such arrangements, the force sensor is arranged to measure a force imparted to the prosthetic portion 300, whether the force is imparted to the surface thereof, through the body of the prosthetic portion 300, to the interface between the prosthetic portion 300 and the anchor portion 200, or to the anchor portion 200 by way of the prosthetic portion 300. In other aspects, the sensor device 400, in the configuration of a force sensor, is engaged with the anchor portion 200 and arranged to measure a force imparted to the anchor portion 200.

In some instances, the anchor portion 200 defines external threads adapted to threadedly engage a structure such as, for example, the jaw of the patient in a previously-formed borehole. In such instances, the force sensor 420 is engaged with or is otherwise in communication with the external threads and is arranged to measure a threading force engaging the anchor portion 200 with the structure. Such an arrangement can measure, for example, whether the anchor portion 200 is securely seated in the jawbone upon being implanted, whether the anchor portion 200 is progressing toward or has attained suitable osseintegration with the jawbone, and/or whether the threads of the anchor portion 200 have loosened or otherwise may be indicating an endpoint of service life.

In other instances, in addition to or in an alternative to the force sensor 420 engaged with the external threads of the anchor portion 200, the anchor portion 200 may be arranged to include a wall 430 defining an internal channel (e.g., an internal hollow channel extending along the length of the anchor portion 200), and a force sensor 440 can be arranged to be engaged with the wall 430 within the internal channel and is further arranged to measure forces imparted to the anchor portion 200. A force sensor 440 arranged in this manner may indicate, for example, a bending moment exerted on the anchor portion 200.

In some aspects, the sensor device 400 is a contact sensor 460 (e.g., a force sensor, a pressure sensor, a touch sensor, and/or suitable surface sensor) engaged with a surface of the prosthetic portion 300 and is arranged to sense external contact with the prosthetic portion 300. A contact sensor 460 arranged in such a manner on a surface of the prosthetic portion 300 can indicate, for example, locations of occlusion across the surface that allows the prosthetic portion 300 to be adjusted, re-shaped, etc. in order to minimize or eliminate the localized occlusion.

In other aspects, the sensor device 400 is a force sensor or a contact sensor 480 engaged with the anchor portion 200 or the prosthetic portion 300, or otherwise disposed between the anchor portion 200 and the prosthetic portion 300. The force/contact sensor 480 arranged in this manner can indicate, for example, the security of the connection between the anchor portion 200 and the prosthetic portion 300, force(s) experienced by the anchor portion 200 by way of the prosthetic portion 300, and/or the number of times that a threshold force is experienced by the anchor portion 200 and/or the prosthetic portion 300 by way of force(s) experienced by the prosthetic portion 300.

In some aspects, the dental prosthetic device 100 further comprises a power source 500 arranged in electrical communication with the sensor device 400, and disposed within the anchor portion 200. In other aspects, particularly where the dental prosthetic device 100 comprises the anchor portion 200 and the prosthetic portion 300, the power source 500 is arranged in electrical communication with the sensor device 400, and disposed within the anchor portion 200, within the prosthetic portion 300, or between the anchor portion 200 and the prosthetic portion 300. In particular instances, the power source 500 is configured and arranged to as to have an extended service life, or to be capable of generating electrical power, without the power source 500 requiring recharging from an external source or without replacement of the power source 500 in the dental prosthetic device 100. For example, in such instances, the power source 500 can be arranged to generate electrical power in response to vibration or movement (e.g., while the patient is chewing). In other instances, the power source 500 can include a battery 520, as well as a charging device 540 in electrical communication with and configured to charge/recharge the battery 520, wherein the charging device 540 may be configured and arranged to generate electrical power for charging/recharging the battery 520, in response to vibration or movement. The power source 500 is therefore arranged to provide electrical power at least to the sensor device 400.

In other aspects, the dental prosthetic device 100 can include at least a transmitter 600 in communication with the sensor device 400 and with the power source 500. In particular instances, the transmitter 600 is housed by or otherwise engaged with the anchor portion 200. In other instances, the transmitter 600 is housed by or otherwise engaged with the anchor portion 200, with the prosthetic portion 300, or between the anchor portion 200 and the prosthetic portion 300. In such instances, the transmitter 600 is arranged to transmit data collected from the sensor device 400 away from the dental prosthetic device 100. For example, the data can be transmitted from the dental prosthetic device 100 to a receiver 700 at a discrete/separate analysis station such that the data can be analyzed in regard to parameter(s) sensed by the particular type(s) of sensor(s) included in the sensor device 400. For example, the transmitter 600 can be configured and arranged to transmit the data via Bluetooth, Near-Field Communication (NFC), WiFi, or any other suitable wireless or contactless communication method, wherein the communication with the receiver 700 requires the receiver 700 to be appropriately configured to communicate with the transmitter 600 and receive the data therefrom.

In some aspects, the transmitter 600 is configured to implement a communication modality such as, for example, NFC, inductive data transmission, electromagnetic data transmission, ultrasonic data transmission, or other suitable protocol, wherein the transmitter 600 is electrically powered or is active on a limited basis (e.g., on demand or at selected time intervals) in order to reduce power requirements. For example, the transmitter 600 configured for NFC data transmission can normally be in sleep mode or otherwise in a dormant state (e.g., not transmitting data and not otherwise consuming power from the power source 500). However, when brought into proximity to a receiver 700 similarly configured for NFC and to initiate contactless communication (e.g., a contactless communication modality) therebetween, the proximity between the transmitter 600 and the receiver 700 wakens or otherwise activates the transmitter 600 for the receiver 700 to capture sensor data therefrom, thereby reducing, minimizing, or eliminating the power requirement for the transmitter 600 from the power source 500.

Moreover, the receiver 700 at the analysis station can be arranged and configured, for example, in conjunction with an application running on a mobile device, or a suitable computer program product executed on a computer device having a processor and a memory storing executable instructions executable by the processor for performing the data analysis. Based on the parameter(s) being monitored by the sensor device 400, the application on the mobile device and/or the computer program product on the computer device can be arranged/configured to actuate an alarm or other suitable warning if a parameter sensed by the sensor device 400 is outside of threshold limit(s) such that appropriate remedial action can be taken (e.g., an alert can be actuated in the event of the anchor portion 200 loosening in the jawbone, or when an incident of concern (e.g., excessive force sensed) has been detected).

In some instances, the dental prosthetic device 100 according to various aspects may further include a receiver 650 in addition to the transmitter 600, wherein the receiver 650 may be configured and arranged to receive incoming signals, for example, for actuating various components within the dental prosthetic device 100. For example, in some aspects, a reservoir 800 is arranged to contain a medicament, and a dispensing device 900 is arranged in communication with the reservoir 800 and arranged and configured to dispense the medicament from the reservoir 800 away from the dental prosthetic device 100. The reservoir 800 and the dispensing device 900 can be disposed within the anchor portion 200. In other aspects where the dental prosthetic device 100 includes a prosthetic portion 300, each of the reservoir 800 and the dispensing device 900 can be disposed within the anchor portion 200, within the prosthetic portion 300, or between the prosthetic portion 300 and the anchor portion 200. In such an arrangement, the dispensing device 900 can be arranged in communication with the receiver 650, and to be responsive to an actuation signal received via the receiver 650 to release the medicament from the reservoir 800 and outwardly of the dental prosthetic device 100. For example, the medicament can be released from the reservoir 800 into the patient's bloodstream upon the dispensing device 900 receiving an actuation signal via the receiver 650. In some instances, instead of or in addition to a medicament, the reservoir 800 can contain, for example, nanoscale bioelectronics which can be released into the patient's bloodstream, as necessary, so as to facilitate, for example, blood analysis (e.g., cancer screening) or other biological/chemical function.

In other aspects, the dental prosthetic device 100 can include a controller 1000 instead of or in addition to the receiver 650. The controller 1000 can include a timer, appropriate logic, or communication provisions with the receiver to generate an actuation signal to the dispensing device 900 for releasing the medicament, bioelectronics, and/or other substance (e.g., an anesthetic) into the patient's bloodstream. In some instances, the controller 1000 is arranged and configured to receive and analyze data from the sensor device 400, and then to take appropriate action based upon the data analysis or to then transmit the data and/or analysis of that data outwardly of the dental prosthetic device 100 via the transmitter 600.

Another aspect of the present disclosure is directed to a method of forming a dental prosthetic device having a prosthetic portion arranged to be engageable with an anchor portion. Such a method comprises, for example, engaging a sensor device with an anchor portion of the dental prosthetic. In other aspects, such a method comprises engaging a sensor device with an anchor portion of the dental prosthetic, with the prosthetic portion of the dental prosthetic, or between the prosthetic portion engaged with the anchor portion. Other method aspects associated with the forming of such a dental prosthetic device are readily evident from the disclosure of such a device as otherwise provided herein.

In light of ever-increasing miniaturization of various sensors (e.g., force-measuring film sensors) and other electronic components, aspects of the present disclosure provide an enhanced dental implant/prosthetic device that incorporates these miniaturized sensors and other miniaturized electronic components into the device so as to provide an active, “intelligent” device capable or sensing, monitoring, analyzing, and remedying conditions that can occur during the service life of the device.

Aspects of the dental prosthetic device according to the disclosure are thus capable of indicating whether the dental implant has been properly implanted in the patient's jawbone, and whether the patient's jawbone has accepted and properly osseointegrated the anchor portion. Other aspects of the dental prosthetic device are also capable of indicating the various force(s) experienced by the dental implant in service such that possible damage to the dental implant can be investigated and remedied prior to failure, and allow for the monitoring force(s) and force cycle(s) experienced by the dental implant such that remedial action before failure can be taken prior to the dental implant reaching fatigue limits or end of service life.

Many modifications and other embodiments of the inventions set forth herein will come to mind to one skilled in the art to which these disclosed embodiments pertain having the benefit of the teachings presented in the foregoing descriptions and the associated drawings. Therefore, it is to be understood that embodiments of the invention are not to be limited to the specific embodiments disclosed and that modifications and other embodiments are intended to be included within the scope of the invention. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the disclosure. In this regard, for example, different combinations of elements and/or functions than those explicitly described above are also contemplated within the scope of the disclosure. Although specific terms are employed herein, they are used in a generic and descriptive sense only and not for purposes of limitation.

It should be understood that although the terms first, second, etc. may be used herein to describe various steps or calculations, these steps or calculations should not be limited by these terms. These terms are only used to distinguish one operation or calculation from another. For example, a first calculation may be termed a second calculation, and, similarly, a second step may be termed a first step, without departing from the scope of this disclosure. As used herein, the term “and/or” and the “/” symbol includes any and all combinations of one or more of the associated listed items.

As used herein, the singular forms “a”, “an” and “the” are intended to include the plural forms as well, unless the context clearly indicates otherwise. It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

That which is claimed:

1. A dental prosthetic device, comprising:
an anchor portion; and
a sensor device arranged to be engaged with the anchor portion.

2. The device of claim 1, comprising a prosthetic portion arranged to engage the anchor portion, wherein the sensor device is arranged to be engaged with the anchor portion, with the prosthetic portion, or between the anchor portion and the prosthetic portion.

3. The device of claim 1, wherein the sensor device is a force sensor.

4. The device of claim 2, wherein the sensor device is a force sensor, and wherein the force sensor is disposed between the anchor portion and the prosthetic portion, within the prosthetic portion, or on a surface of the prosthetic portion, and is arranged to measure a force imparted to the prosthetic portion.

5. The device of claim 3, wherein the anchor portion defines external threads adapted to threadedly engage a structure, and wherein the force sensor is engaged with the external threads and is arranged to measure a threading force engaging the anchor portion with the structure.

6. The device of claim 3, wherein the anchor portion includes a wall defining an internal channel, and wherein the force sensor is engaged with the wall within the internal channel and is arranged to measure forces imparted to the anchor portion.

7. The device of claim 2, wherein the sensor device is a contact sensor engaged with a surface of the prosthetic portion and is arranged to sense external contact with the prosthetic portion.

8. The device of claim 1, comprising a power source arranged in electrical communication with the sensor device, and disposed within the anchor portion.

9. The device of claim 2, comprising a power source arranged in electrical communication with the sensor device, and disposed within the anchor portion, within the prosthetic portion, or between the anchor portion and the prosthetic portion.

10. The device of claim 1, comprising a power source arranged in electrical communication with the sensor device, wherein the power source is arranged to generate electrical power in response to vibration or movement thereof.

11. The device of claim 10, comprising a transmitter in communication with the sensor device and with the power source, wherein the transmitter is arranged to be powered by the power source to transmit data from the sensor device away from the dental prosthetic.

12. The device of claim 11, comprising a prosthetic portion arranged to engage the anchor portion, wherein the transmitter is engaged with the anchor portion, with the prosthetic portion, or between the anchor portion and the prosthetic portion.

13. The device of claim 1, comprising a transmitter in communication with the sensor device, wherein the transmitter is arranged to implement a contactless communication modality to transmit data from the sensor device away from the dental prosthetic.

14. The device of claim 1, comprising a reservoir arranged to contain a medicament, and a dispensing device in communication with the reservoir and arranged to dispense the medicament therefrom away from the dental prosthetic.

15. The device of claim 14, comprising a prosthetic portion arranged to engage the anchor portion, wherein each of the reservoir and the dispensing device is arranged within the anchor portion, within the prosthetic portion, or between the prosthetic portion and the anchor portion.

16. A method of forming a dental prosthetic device, comprising:
engaging a sensor device with an anchor portion of a dental prosthetic.

17. The method of claim 16, wherein the dental prosthetic includes a prosthetic portion arranged to be engageable with the anchor portion, and wherein the method comprises engaging the sensor device with the anchor portion, with the prosthetic portion, or between the prosthetic portion and the anchor portion.

18. The method of claim 16, wherein engaging the sensor device comprises engaging a force sensor with the anchor portion.

19. The method of claim 17, wherein engaging the sensor device comprises engaging a force sensor between the anchor portion and the prosthetic portion, within the prosthetic portion, or on a surface of the prosthetic portion, such that the force sensor is arranged to measure a force imparted to the prosthetic portion.

20. The method of claim 18, wherein the anchor portion defines external threads adapted to threadedly engage a structure, and wherein engaging the sensor device comprises engaging the force sensor with the external threads such that the force sensor is arranged to measure a threading force engaging the anchor portion with the structure.

21. The method of claim 18, wherein the anchor portion includes a wall defining an internal channel, and wherein engaging the sensor device comprises engaging the force sensor with the wall within the internal channel such that the force sensor is arranged to measure forces imparted to the anchor portion.

22. The method of claim 17, wherein engaging the sensor device comprises engaging a contact sensor with a surface of the prosthetic portion such that the contact sensor is arranged to sense external contact with the prosthetic portion.

23. The method of claim 16, comprising arranging a power source within the anchor portion such that the power source is in electrical communication with the sensor device.

24. The method of claim 17, comprising arranging a power source within the anchor portion, within the prosthetic portion, or between the anchor portion and the prosthetic portion, such that the power source is in electrical communication with the sensor device.

25. The method of claim 16, comprising arranging a power source in electrical communication with the sensor device, wherein the power source is arranged to generate electrical power in response to vibration or movement thereof.

26. The method of claim 25, comprising arranging a transmitter in communication with the sensor device and with the power source, wherein the transmitter is arranged to be powered by the power source to transmit data from the sensor device away from the dental prosthetic.

27. The method of claim 26, wherein the dental prosthetic includes a prosthetic portion arranged to engage the anchor portion, and wherein arranging the transmitter comprises arranging the transmitter in engagement with the anchor portion, with the prosthetic portion, or between the anchor portion and the prosthetic portion.

28. The method of claim 16, comprising engaging a transmitter in communication with the sensor device, wherein the transmitter is arranged to implement a contactless communication modality to transmit data from the sensor device away from the dental prosthetic.

29. The method of claim 16, comprising engaging a reservoir arranged to contain a medicament, and a dispensing device in communication with the reservoir and arranged to dispense the medicament therefrom away from the dental prosthetic, with the anchor portion.

30. The method of claim 29, wherein the dental prosthetic includes a prosthetic portion arranged to engage the anchor portion, and wherein the method comprises arranging each of the reservoir and the dispensing device within the anchor portion, within the prosthetic portion, or between the prosthetic portion and the anchor portion.
